(19) United States
(12) Patent Application Publication
(10) Pub. No.: US 2017/0226246 A1
(43) Pub. Date: Aug. 10, 2017

(54) CATIONIC PARTICLES COMPRISING CYCLOPROPENIUM, THEIR PREPARATION AND USES

(71) Applicants: The Trustees of Columbia University in the City of New York, New York, NY (US); U.S. Army Edgewood Chemical Biological Center, Aberdeen Proving Ground, MD (US)

(72) Inventors: Kathryn Louise Killops, Beaverton, OR (US); Luis M. Campos, Brooklyn, NY (US); Spencer D. Brucks, New York, NY (US)

(21) Appl. No.: 15/502,665

(22) PCT Filed: Aug. 7, 2015
(86) PCT No.: PCT/US15/44331

§ 371 Date: Feb. 8, 2017

Related U.S. Application Data
(60) Provisional application No. 61/999,886, filed on Aug. 8, 2014.
(60) Provisional application No. 62/169,859, filed on Jun. 2, 2015.

(51) Int. Cl. C08F 212/08; C09B 69/10; C08F 293/00; A61K 48/00; G01N 33/58

(52) U.S. Cl. CPC C08F 212/08; A61K 48/0041; G01N 33/587; G01N 33/582; C08F 293/005; C09B 69/106

(57) ABSTRACT

Embodiments of the present invention provides compounds, compositions, and methods for their preparation or synthesis that provide polymer-based cationic particles, such as, e.g., polymer-nucleic acid complexes, for delivering molecules including biomolecules, which is particularly desirable in gene therapy. Inventive materials include positively-charged linear homopolymers and block copolymers by living free radical polymerization, and polymer-based particles by emulsion polymerization. These polymers and particles may be conjugated with a wide range of biomolecules, and may deliver molecules, including, drug molecules, contrast agents, dyes, and the like, by loading them into the interior of the particles prior to polymerization. These conjugated and/or labeled polymers and particles may be delivered to cells to administer their cargo and achieve a therapeutic response. Additional embodiments may be directed to the methods of synthesizing and using the compounds and compositions, as well as kits comprising the compounds, compositions, and formulations, and desired molecules for delivery.

### CROSS-REFERENCES TO RELATED APPLICATIONS

This international PCT Application claims the benefit of priority from U.S. Provisional Patent Application No. 61/999,886, filed Aug. 8, 2014, entitled, “Particles Comprising Cyclopropenium, Their Preparation And Uses,” and U.S. Provisional Patent Application No. 62/169,859, filed Jun. 2, 2015, entitled, “New Materials for Gene-Delivery to Cancer Cell Lines that are Difficult to Transfect,” which are incorporated here by reference in their entirety.

### STATEMENT REGARDING FEDERALLY SPONSORED RESEARCH OR DEVELOPMENT

The present invention was made with government support under contract number W911NF-12-1-0252 awarded by the Army Research Office. The United States government may have certain rights in this invention.

### TECHNICAL FIELD

The invention relates generally to carriers. In particular, the present invention relates to positively-charged polymer-based particles of cyclopropenium-containing molecules for delivering a desired molecule.

### BACKGROUND

Cationic nanoparticles are of interest in a diverse range of fields from gene-based therapeutics to cosmetics to drug delivery. The preparation of polymer-based cationic or polycationic particles can be achieved by a number of different strategies Including aggregation and crosslinking of linear polymers, formation and crosslinking of micelles, and polymerization under confinement as in an emulsion.

Cationic polymers and particles have been envisaged as substrates or carriers to deliver a biological or chemical or active “cargo” to cells in vivo via complexation to the cargo. Specifically, the development of polymer-based cationic particles has focused on their use as non-viral vectors for gene therapy. Often, a carrier is needed to deliver the genes through the cell's cytoplasm and help protect them from enzymatic degradation. The positive charge exuded by the polymers/particles makes them well-positioned for electrostatic binding to negatively charged polynucleic acids (PNA: DNA, mRNA, siRNA, etc.). Many conventional technologies containing positively charged groups are derived from pH sensitive protonated or quaternated amines.^{10,11} Therefore, fluctuations in pH may cause the charged group to become deprotonated, affecting the colloidal stability and attachment of the cargo.⁸ The delivery of genetic material to cells to regulate genetic expression or interfere with unfavorable cellular processes is highly desirable for next-generation therapeutics. The opportunity for low cost and tunable structures afforded by a polymer-based gene transfection platform is highly advantageous for the advancement and accessibility of genetic therapeutics.¹²

The ability of a substrate to deliver genetic cargo to cells is contingent on the stabilization of the PNA imparted by the particle and the inclusion of targeting moieties to deliver the cargo to the cells of interest.^{13,14} Current non-viral gene transfection agents include cationic lipids¹⁵ (Lipofectamine®, iFect), polyamines¹⁶ (poly(L-lysine), polyethylenimine (PEI), polyamidoamine (PAMAM)), and polysaccharide (chitosan), among others.^{17,19} There are a number of issues with conventional technologies, which suffer from a number of limitations, including cytotoxicity, pH sensitivity, difficulty of synthesis, targeting and delivery, lack of varability, and polynucleic acid (PNA) release. There is a great need for a delivery system that has pH stability, modularity, and control over size or charge, among others, which can help overcome some of the disadvantages of these conventional systems.

### SUMMARY

The invention relates to cationic polymer/particle complexes that may be used as a carrier to deliver an active ingredient. For example, nucleic acids for gene therapy may be delivered in a polymer-nucleic acid complex. In particular, the cationic particles are desirable because they overcome a multitude of limitations that conventional delivery systems suffer. The synthesis, conjugation, and delivery of cationic polyplexes based on linear polymers and latex particles to cells are included. The invention may be applicable to a range of fields including, preferably, gene delivery, drug delivery, diagnostics, enzyme stabilization, therapeutics, filtration/separation, cosmetics, imaging, viscosity modifiers, and coatings, among others.

Moreover, the polycationic particles, such as for example, poly-trisaminocyclopropenium ions are simple to prepare, broadly tunable in terms of their properties, and are stable in pH ranging from about 0 to greater than about 11. These stable poly-cationic particles offer unparalleled performance for drug delivery, DNA binding, imaging, diagnostics, and a myriad of other applications. Preferably, the invention may be directed to non-viral gene transfection agents based on the cyclopropenium polymers for gene delivery.

The cyclopropenium-containing moiety described here is exceptional compared to competing or conventional materials because it is stable, but also maintains its positive charge at pH values as high as pH 11. Whereas, conventional competitor materials use basic units that are protonated (such as for example, amines) and those lose their charge at pH above 7. Due to the loss of charge at physiological conditions, the conventional materials are less efficient in their respective applications.

Other advantages of the disclosed invention include its modularity. The emulsion polymerization strategy also enables the incorporation of functionality both in the interior of the particle (i.e., hydrophobic dyes) and enables conjugation at the periphery (i.e., charged biomolecules, DNA). The synthesis produces cationic latexes that can maintain their charge at high pH, and definitely at physiological levels.

### BRIEF DESCRIPTION OF THE DRAWING FIGURES

The patent or application file contains at least one drawing executed in color. Copies of this patent or patent application publication with color drawing(s) will be provided by the Office upon request and payment of the necessary fee.

FIG. 1 shows the results of a DNA binding assay by gel electrophoresis for (A) a cyclopropenium-based polymer; and (B) cationic particles. Ratios are provided as cyclopropenium monomers to DNA nucleotides.

FIG. 2 shows the synthesis of particles by emulsion polymerization using water-soluble azo initiator.

FIG. 3 shows data on particles synthesized with 10 wt. % cyclopropenium monomer. (A) Scanning probe microscopy image of dry particles; (B) electrophoretic mobility measurement of particles in water; and (C) size distribution from dynamic light scattering (DLS).

FIG. 4 shows a scanning electron microscope image of particles based on (a) styrene only, and (b) with 1CPiP (see Table 1). Size distribution data (c) obtained by dynamic light scattering of nanoparticles synthesized with varied weight percent incorporation of CPiP relative to styrene (see TABLE 1 for details). Scale bars are 200 nm. Curves from left to right represent 20 CPiP to 1 CPiP, respectively.

FIG. 5 shows scanning electron microscope images of particles synthesized with 95% styrene and 5% CP monomer by weight: (a) CPip, (b) CPCy, and (c) CPMo. All scale bars are 200 nm.

FIG. 6 shows Zeta-potential as a function of pH with 5CPiP particles (circles). Particle radii were measured simultaneously (triangles).

FIG. 7 shows normalized excitation (black) and emission (red) spectra of CPiP particles synthesized with (a) FMA (πₑₓ 500) or (b) PMA (λₑₓ 345). The gray dashed line indicates the absorption spectrum of 10CPiP particles without dye.

FIG. 8 shows SEM images of particles synthesized with CP BCPs (a) 5iPBCP, (b) 5CyBCP, and (c) 5MoBCP. All scale bars are 200 nm.

FIG. 9 shows an illustrated representation of the surfactant-free emulsion polymerization of (a) CPR Monomers and (b) PS-b-PCPR BCPEs with styrene to form surface-charged polymer nanoparticles.

FIG. 10 shows ¹H NMR spectrum of lyophilized 20iPM particles dissolved in CDCl₃.

FIG. 11 shows UV Vis spectra of Congo Red dye before and after incubation with either BCPE- or monomer-derived particles. The reduction in absorbance corresponds to a 41% decrease in dye concentration upon incubation with BCPE particles and a 48% decrease in dye concentration for monomer-derived particles.

FIG. 12 shows exemplary monomers typically used in cationic latexes.

FIG. 13 shows particles with modular functionality. 5 wt. % CPR incorporation. A) CPCy: Dₙ=62±1 nm, Nₚ=3.17×10¹⁷ L⁻¹, ζ_{pH=7}=21±1 mV. B) CPiP: Dₙ=59±1 nm, Nₚ=7.17×10¹⁷ L⁻¹, ζ_{pH=7}=26±2 mV. C) CPMo: Dₙ=166±8 nm, Nₚ=1.59×10¹⁶ L⁻¹, ζ_{pH=7}=14±1 mV.

FIG. 14 shows particles maintaining their charge with pH. 10% CPCy particles with AIBN. pK^{R+}≧13 for triaminocyclopropeniums. Properties of cationic particles with charged heteroatoms are highly contingent on pH. Zeta potential stays highly positive over a large pH range.

FIG. 15 shows CP incorporation affects particle size. Particle diameter decreases, while zeta potential and polydispersity index (PDI) increase with increasing CPiP incorporation. A) Size distribution data obtained by dynamic light scattering of nanoparticles synthesized with varied weight percent incorporation of CPiP relative to styrene. B) Zeta-potential as a function of weight percent CPiP incorporation. C) Hydrodynamic diameter (Dₕ) and Polydispersity index (PDI) corresponding to weight percent CPiP.

FIG. 16 shows that Nₚ increases with incorporation of CPiP, which is nearly exponential above 1%.

FIG. 17 shows CPR BCPs as particle stabilizers. Stabilized particles formed at 5 wt % CPR BCP. A) PS-b-P (CPiP): Dₕ=94±2 nm, PDI=0.15±0.03, ζ=39±1 mV. B) PS-b-P (CPCy): Dₕ=138±1 nm, PDI=0.11±0.01, ζ=22±5 mV.

FIG. 18 shows the encapsulation of dye molecules. A) Nile Red and B) Coumarin 153.

FIG. 19 shows binding of dyes to particle surface. A) and C) provide structures of exemplary dyes. B) and D) demonstrate binding of dye to particle surface, where (B) from left to right the gradient of colors darkens from a cloudy yellow to a dark orange, and (D) a cloudy pink to a dark pink.

FIG. 20 shows DNA binding with CPiP particles. A) Phosphates on DNA backbone interact with CPiP particles. B) PS-b-PEO control.

FIG. 21 shows DNA binding with CPMo particles. A) Phosphates on DNA backbone interact with CPMo particles. B) PS-b-PEO control.

### DETAILED DESCRIPTION OF THE INVENTION

Embodiments of the present invention describe the synthesis of polymer-based particles by emulsion polymerization. Other embodiments may be directed to the synthesis of positively-charged linear homopolymers and block copolymers (BCPs) by living free radical polymerization, The polymers and particles can be conjugated with a wide range of biomolecules, primarily by electrostatic interactions to form polyplexes. Additionally, cargo (e.g., drug molecules, contrast agents, dyes, etc.) can be loaded into the interior of the particles prior to polymerization. These conjugated and/or labeled polymers and particles can be delivered to cells to administer their cargo and achieve a therapeutic response.

One embodiment of the invention may be directed to a process for the synthesis of cationic nanoparticles, preferably cationic surface-charged nanoparticles, containing cyclopropenium-based chemical moieties, where the synthesis takes place by oil-in-water emulsion polymerization, water-in-oil emulsion polymerization, a seeded emulsion polymerization; where the cyclopropenium may be contained in block copolymers that are used to stabilize the emulsion and incorporated into the particles, or in a branched or dendritic polymeric architecture that is used to stabilize the emulsion and incorporated into the particles; where the particles contain co-monomer species including, but not limited to, styrenic, acrylic, methacrylic, and the like. A multivalent crosslinking component may also be added. Nanoparticles generally have a diameter ranging from about 10 nm to about 500 nm.

Embodiments of the invention describe the synthesis of positively-charged polymer-based particles by emulsion polymerization of cyclopropenium-containing molecules. The particles generally maintain their charge over a wide pH range (i.e., about 1 to about 11). (see, FIG. 14) The particles can act as a stabilizing support to prevent the degradation or denaturization of molecules, particularly biomolecules such as oligonucleotides and enzymes. Additionally, cargo, such as, for example, drug molecules, contrast agents, dyes, and the like, can be loaded into the interior of the particles prior to polymerization. These conjugated and/or labeled particles can be delivered to cells to administer their and achieve some therapeutic response. Furthermore, the incorporation of dyes, fluorescent labels, or contrast agents could be useful in imaging and diagnostic applications. Moreover, the positively-charged polycationic particles should enable a broad range of applications, including but not limited to drug delivery, gene or drug delivery, ion-exchange resins, affinity binding or chromatography (DNA, proteins), filtration/purification, immunoassays, enzyme stabilization, antimicrobial coatings/antibiofouling, organocatalyst supports, cosmetics, therapeutics, diagnostics, and the like.

### Methods of Synthesizing

In one embodiment, the cationic particles comprising cyclopropenium are prepared or synthesized by emulsion polymerization. Cyclopropenium cations, having the smallest Hückel aromatic structure, are stable, persistent cations insensitive to pH change (pK_{R+} greater than or equal to 13 for triaminocyclopropeniums), and stable to oxidation. Emulsion polymerization is a method for producing polymers that utilize a continuous liquid phase in which a discontinuous liquid phase is dispersed. Monomers polymerize in water which essentially acts as the continuous phase, and the monomer presents itself in monomer droplets in the water. Basically, the monomers are polymerized in micelles that are formed by surfactant. A water-soluble initiator starts the reaction. After the reaction has completed, the resulting product is a latex that comprises a colloidal dispersion of the polymer particles in water. One advantage of emulsion polymerization is the combination of properties of two or more monomers into one polymer. This copolymerization occurs by polymerizing a first monomer to form a seed latex and then further polymerizing the seed latex with the other monomer to make polymer chains with the desired properties.

Emulsion polymerization is a type of radical polymerization, allows for the incorporation of functionality in the interior of the particle, and also enables conjugation at the periphery. Emulsion polymerization may involve dispersing monomers in water with surfactants and a water-soluble initiator. Cationic surfactants, such as for example, cetyltrimethylammonium bromide (CTAB) and dodecyltrimethylammonium bromide (DTAB), are non-covalently bound and can desorb. Cationic particles, preferably nanoparticles, may be synthesized by surfactant-free emulsion polymerization through the use of cationic co-monomers or initiators. Examples of these monomers typically used in cationic latexes are shown in FIG. 12. The cationic initiators used in this embodiment preferably have two charges per chain at the most. The resulting particles are pH sensitive as the pH alters the charge on protonated species. Additionally, quaternated heteroatoms lack synthetic versatility.

In one embodiment, the cationic particles may be synthesized by surfactant-free emulsion polymerization using cationic co-monomers or initiators. More specifically, one embodiment of the invention is directed to surfactant-free emulsion polymerization for synthesizing cationic surface-charged nanoparticles using cyclopropenium (CP)-based monomers and block copolyelectrolytes (BCPEs). Using novel CP-based monomers and block copolyelectrolytes, cationic polymer nanoparticles may be synthesized, and the two systems may be used as interfacial stabilizers. The propensity of CP-containing molecules to stabilize the particle interface eliminated the need for additional surfactants, solvent mixtures, or multistep protocols, while enabling the formation of uniform sub-100 nm particles. Particle sizes of sub-100 nm are particularly promising for in vitro and in vivo applications (Elsabahy, M.; Wooley, K. L. Chem. Soc. Rev. 2012, 41, 2545; Minigo, G.; Scholzen, A.; Tang, C. K; Hanley, J. C.; Kalkanidis, M.; Pietersz, G. A.; Apostolopoulos, V.; Plebanski, M. Vaccine 2007, 25, 1316), especially for their use in biomedical technologies. Embodiments of the invention are directed to positively-charged polycationic nanoparticles having a diameter of less than about 100 nm, preferably about 30 nm to about 100 nm, and more preferably about 70 nm. The hydrodynamic diameters of the resultant particles can be reliably tuned simply by varying the amount of CP monomer added. The particles were found to have highly positive zeta potential values, which were maintained over a wide pH range. (see, FIG. 14) These nanoparticles will have tremendous potential for a range of biological, imaging, and industrial applications. A surfactant-free means for producing nano-objects that are well characterized may be polymerization-induced self-assembly which requires prior synthesis of a macro chain-transfer agent. (Charleux, B.; Delaittre, G.; Rieger, J.; D'Agosto, F. Macromolecules 2012, 45, 6753; Zhang, X.; Boissé, S.; Zhang, W.; Beaunier, P.; D'Agosto, F.; Rieger, J.; Charleux, B. Macromolecules 2011, 44, 4149).

Surfactant-free emulsion polymerization typically synthesizes cationic particles larger than 100 nm. The process of which involves a hydrophobic monomer, such as for example styrene, which is copolymerized with a cationic monomer and a radical initiator. (Liu, Q; Li, Y.; Duan, Y.; Zhou, H. Polym. Int. 2012, 61, 1593) Several conventional methods to develop latex particles in the sub-100 nm range utilize surfactants (Ramos, J.; Costoyas, A.; Forcada, J. J. Polym. Sci, Part A: Polym. Chem. 2006, 44, 4461), which can leach from the particles after synthesis or require extensive purification (Ramos, J.; Forcada, J.; Hidalgo-Alvarez, R. Chem. Rev. 2014, 114, 367). Syntheses of monodisperse, sub-100 nm particles by traditional methods without adding surfactants presents a major challenge because of unstable oil-in-water dispersions that are formed (Zhang, G.; Niu, A.; Peng, S.; Jiang, M.; Tu, Y.; Li, M; Wu, C. Acc. Chem. Res. 2001, 34, 249). Unstable particle agglomeration typically subsequently occurs resulting in high polydispersity.

Embodiments of the invention use CP monomers for electrostatic stabilization of the interface and copolymerization with styrene. Based on the finding that higher loadings of CP correspond to large surface-to-volume ratios and smaller particle size, to demonstrate control over particle size, in one embodiment, the amount of CP monomer that may be incorporated into the monomer fee may be adjusted in a range from about 1 weight percent (wt %) to about 20 wt %. (EXAMPLE 10) Systems using traditional cationic units based on protonated tertiary amines, quaternized ammonium ions, and phosphonium ions, among others, i.e., moieties bearing the formal charge on heteroatoms (Ramos, J.; Forcada, J.; Hidalgo-Alvarez, R. Chem. Rev. 2014, 114, 367; Ni, H.; Yongzhong; Ma, G.; Nagai, M.; Omi, S. Macromolecules 2001, 34, 6577; Yuan, J.; Mecerreyes, D.; Antonietti, M Prog. Polym. Sci. 2013, 38, 1009). Although these systems have been useful, the cationic charge is localized, may exhibit pH dependence, and lack modular functional handles which are in contrast to the desired characteristics of embodiments of the invention.

Non-limiting characteristics cyclopropenium ions such as for example, of tris(dialkylamino)cyclopropenium (CP) ions which are a versatile class of carbon-centered cationic materials that exhibit remarkable stability (Yoshida, Z.; Tawara, Y. J. Am. Chem. Soc. 1971, 93, 2573; Curnow, O.; MacFarlane, D. R.; Waist, K. J. Chem. Commun. 2011, 47, 10248) include: resonance charge delocalization through amino substituents (Kerber, R. C.; Hsu, C.-M. J. Am. Chem. Soc. 1973, 95, 3239), yielding electron-rich, stable cations with high pK_{R+} (Yoshida, Z.-i.; Tawara, Y.; Hirota, S.; Ogoshi, H. Bull Chem. Soc. Jpn. 1974, 47, 797), ability to be functionalized with a range of dialkylamines after robust, efficient, and orthogonal chemistry (Campos, L. M.; Lambert, T. H.; Dell, E. J.; Bandar, J. S., WO 2014/022365 A1). CP ions may have applications as ionic liquids (Curnow, O.; MacFarlane, D. R.; Waist, K. J. Chem. Commun. 2011, 47, 10248), organocatalysts (Bandar, J. S.; Lambert, T. H. J. Am. Chem. Soc. 2012, 134, 5552; Bandar, J. S.; Lambert, T. H. J. Am. Chem. Soc. 2013, 135, 11799; Bandar, J. S.; Sauer, G. S.; Wulff, W. D.; Lambert, T. H.; Vetticatt, M. J. J. Am. Chem. Soc. 2014, 136, 10700), transition-metals ligands (Bruns, H.; Patil, M.; Carreras, J.; Vizquez, A.; Thiel, W.; Goddard, R.; Alcarazo, M. Angew. Chem, Int. Ed. 2010, 49, 3680), and polyelectrolytes (Jiang, Y.; Freyer, J. L; Cotanda, P.; Brucks, S. D.; Killops, K. L.; Bandar, J. S.; Torsitano, C.; Balsara, N. P.; Lambert, T. H.; Campos, L. M. Nat. Commun. 2015, DOI: 10.1038/ncomms6950).

One embodiment relates to the use of polymerizable surfactants or surfmers. The initiator may form radicals which initiates polymerization producing a dispersion of polymer particles in water. The key step in the polymerization process is the nucleation or seed stage. Once stable polymer nuclei are formed and stabilized, they continue growing into the final cationic latex particles. Non-limiting examples of emulsions include an oil-in-water emulsion, a water-in-oil emulsion, and a seeded emulsion.

In one embodiment, syntheses of the particles may utilize initiators, preferably water soluble initiators. Non-limiting examples of initiators include: a thermal initiator, an azo initiator (e.g., AIBN, V-50), a peroxide initiator (e.g., K25208), a radical initiator, a photoinitiator, 2,2-Dimethoxy-2-phenylacetophenone (DMPA) or benzophenone, and a redox initiator. The cyclopropenium monomer may be incorporated at about 1% to about 20% of the total weight of the monomer added to the reaction. A degradable moiety may be incorporated, such as, but not limited to, esters, disulfides, amides, and the like. The degradable moiety can be incorporated within the particle and/or into the block copolymer (BCP) chains. Block copolymers may include block copolyelectrolytes (BCPEs). A metal may be incorporated in the synthesis process, creating a composite particle. Synthesis of cyclopropenium particles may be synthesized using cyclopropenium cation (CPR) monomers or CPR block copolymers (BCPs), which act as stabilizers. Non-limiting examples of particles include CPCy, CPiP, and CPMo.

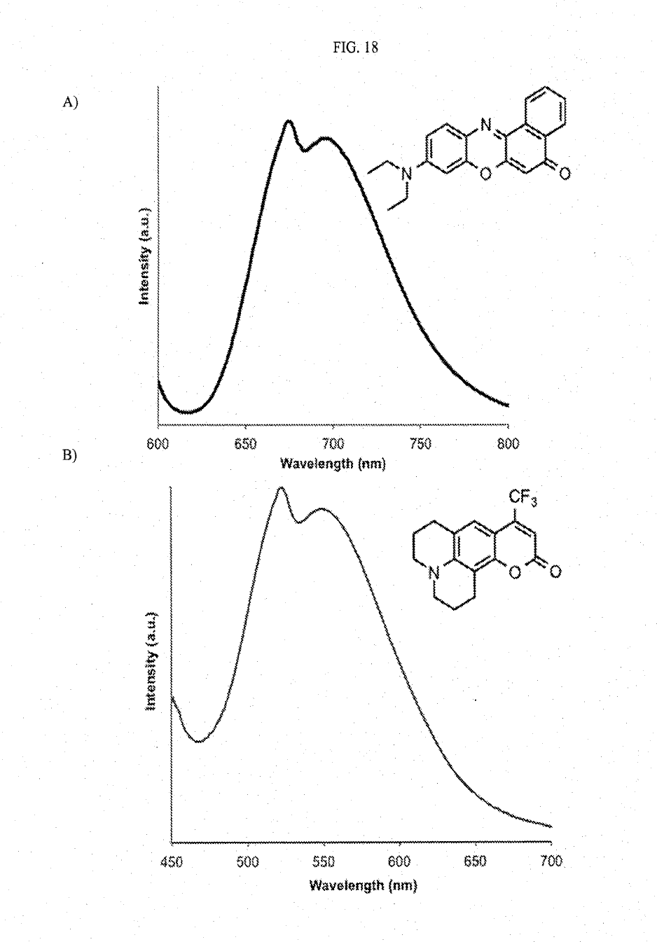

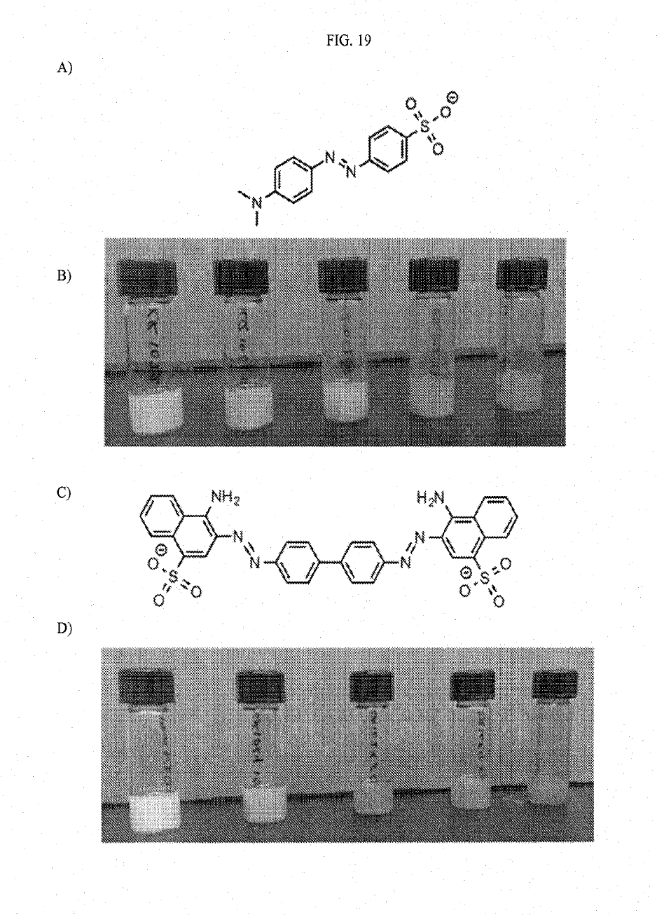

The cargo may include a dye encapsulated in the particle during the synthesis process. The dye may be conjugated to or coated on the exterior of the particle. FIG. 18 shows encapsulation of dye molecules, where hydrophobic dyes were incorporated into the inventive particles. The graphs demonstrate that encapsulation of hydrophobic molecules is possible, and more importantly demonstrates that the nanoparticles may be used for delivering drugs for example. FIG. 19 demonstrates binding of anionic dyes to particle surfaces and flocculation of particles. This also supports the application of dyes with the desired nanoparticles for filtration or water purification for example. In one embodiment, a nucleic acid-based substance, such as but not limited to, mRNA, siRNA, DNA, and the like, may be bound to the particle. Another embodiment may be directed to a protein such as an enzyme that is conjugated to the particle. A further embodiment may be directed to a peptide or other targeting moiety that is conjugated to the particle.

Another embodiment relates to a method for the synthesis of cationic polymers or block copolymers (BCPs) containing cyclopropenium moieties. The cyclopropenium polymer may be linear, block, random, alternating, branched, or the like. In a further embodiment, the cyclopropenium monomer may be copolymerized with styrenic, acrylic, methacrylic, anhydride, other monomer groups, or the like. The polymer may have other polymers grafted to it, such as poly(ethylene glycol). In yet another embodiment, the polymer may have a degradable moiety incorporated such as, but not limited to, esters, disulfides, amides, and the like. The polymer may complexed with a nucleic acid-based substance, such as but not limited to, mRNA, siRNA, DNA, and the like. Another embodiment may be directed to a polymer that has a targeting moiety attached, such as, for example, a peptide.

Embodiments of the invention include the synthesis, conjugation, and delivery of cyclopropenium-based polyplexes to cells. A polymerizable form of cyclopropenium has been published.¹⁹ A unique feature of this cationic moiety is its relative insensitivity to fluctuations in pH. Specifically, this embodiment relates to the use of these cationic polymers/nanoparticles to bind to molecules of interest and deliver them to cells. Without the particles, the molecules of interest could be degraded or rapidly clearedin vivo without ever reaching their target. The particles may also contain drug or dye molecules of interest in their interior, or at the periphery of the particles. Targeting groups, such as peptides, may be incorporated to direct the particles to certain areas in vivo.

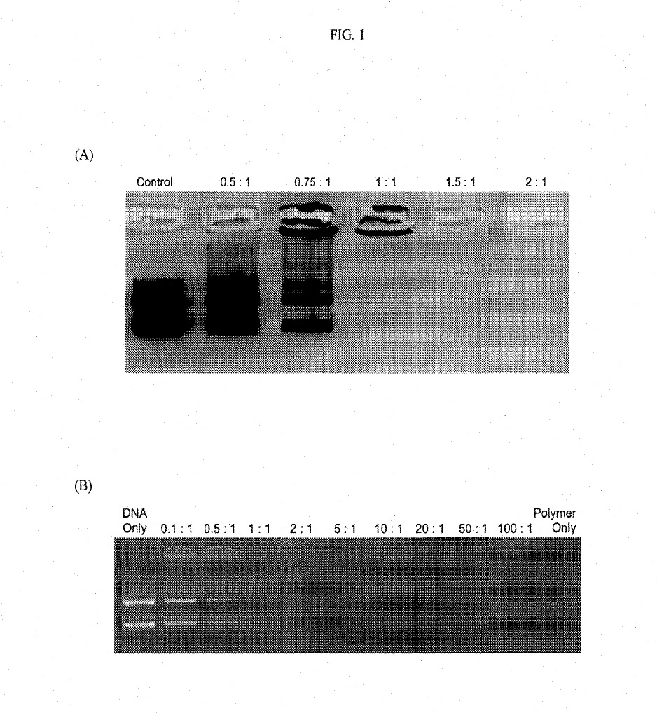

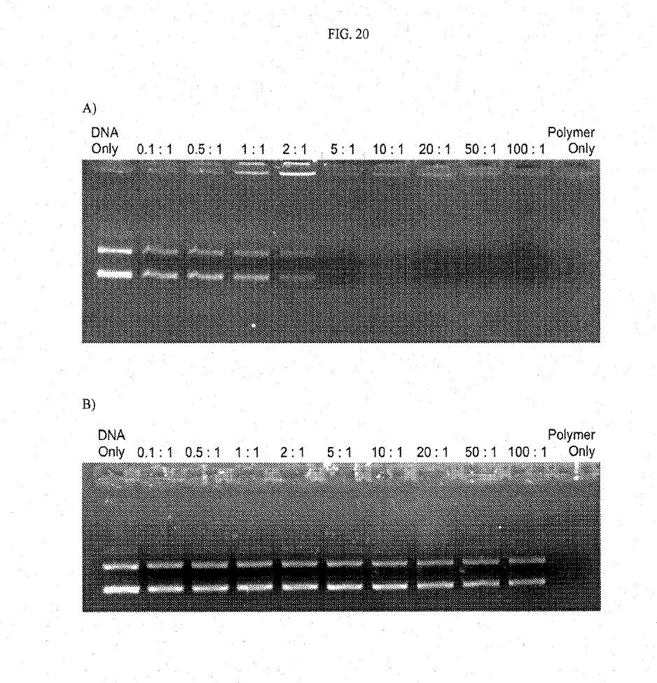

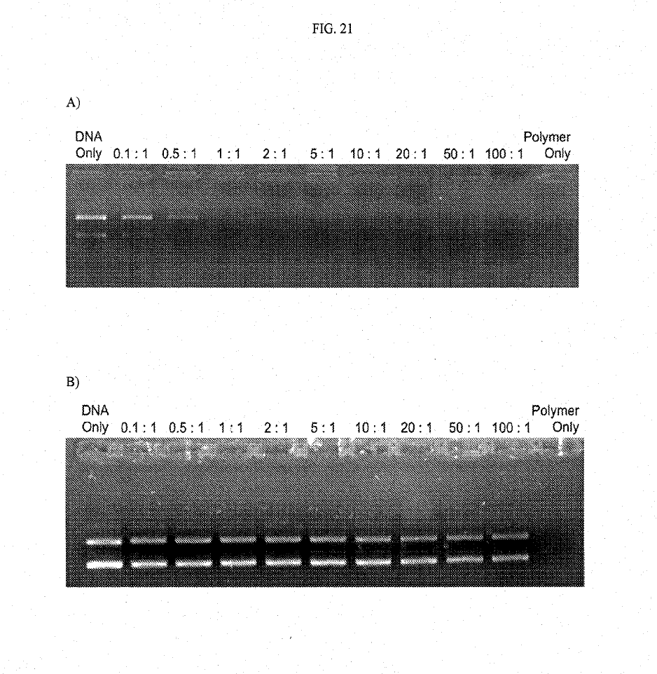

Homopolymers, random copolymers, and block copolymers comprising cyclopropenium have been synthesized using controlled reversible-deactivation radical polymerization techniques. The degree of polymerization (i.e., molecular weight) and incorporation of various components can be precisely controlled. Cyclopropenium-based polymers have been shown to bind DNA effectively, as shown by gel electrophoresis studies (FIG. 1A). The polyplexes formed display a zeta potential near zero and a size of ca. 200 nm by dynamic light scattering. DNA has been shown to bind to cyclopropenium-based polymers at ratios as low as about 1:1 of cyclopropenium monomer units to nucleic acid units. FIG. 20 demonstrates the interaction between phosphates on the DNA backbone and CPiP particles. Complete DNA binding to particles occurred at above 2:1 cation:anion ratio. No non-specific binding was observed for the PS-b-PEO control. FIG. 21 demonstrates the interaction between phosphates on the DNA backbone and CPMo particles. Complete DNA binding to particles occurred at above 0.5:1 cation:anion ratio. About 30 DNA strands bind per particle. No non-specific binding was observed for the PS-b-PEO control. The exemplified binding of DNA to particles confirms that nucleic acids such as RNA may also bind, and the nanoparticles may be applied to gene transfer or delivery.

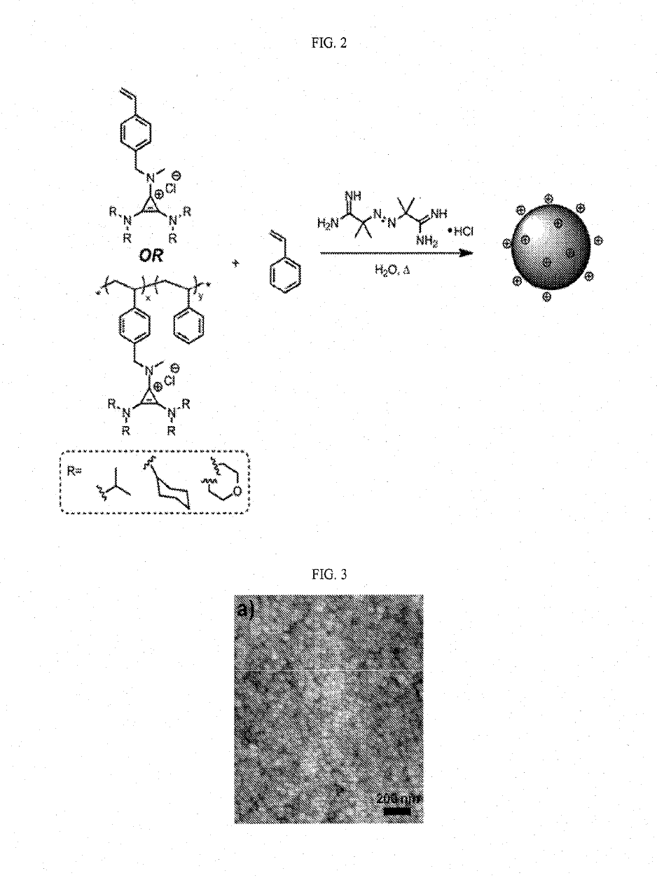

The styrenic cyclopropenium moiety (FIG. 2) has been found to act as an amphiphilic stabilizer for oil-in-water emulsions. Thus, it has been used here to stabilize an emulsion of styrene in water, and with the addition of a radical initiator and heat, polymerization of the stabilized styrene droplets can occur leading to the formation of latex particles comprised of polystyrene and polycyclopropenium. By changing the R groups on the cyclopropenium monomer, different polarities can be achieved. Furthermore, the use of an amphiphilic block copolymer (BCP) containing cyclopropenium groups in one of the blocks (FIG. 2) can also be used to effectively stabilize the emulsion, and incorporated into stable latex particles.

The as-synthesized particles display a highly positive surface charge, as measured by zeta potential. This positive charge is maintained over a wide range of pH values. The positively charged periphery is then used to bind molecules of interest, such as PNA. Conjugating molecules to the particles could have some stabilizing effect that resists cleavage or denaturation. By appending these molecules to a polymer-based support, they may be more effectively delivered in vivo. The particle-based platform can be tailored for different applications by incorporating dyes for tracking or peptides for targeting.

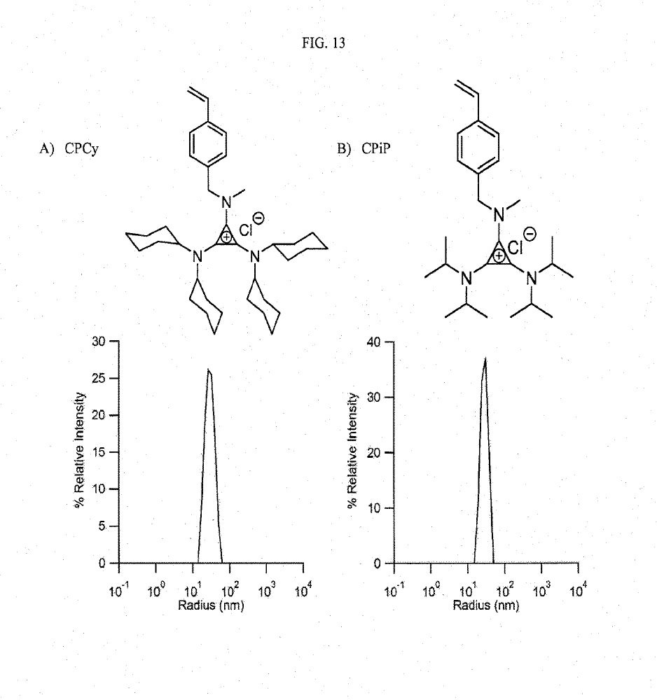

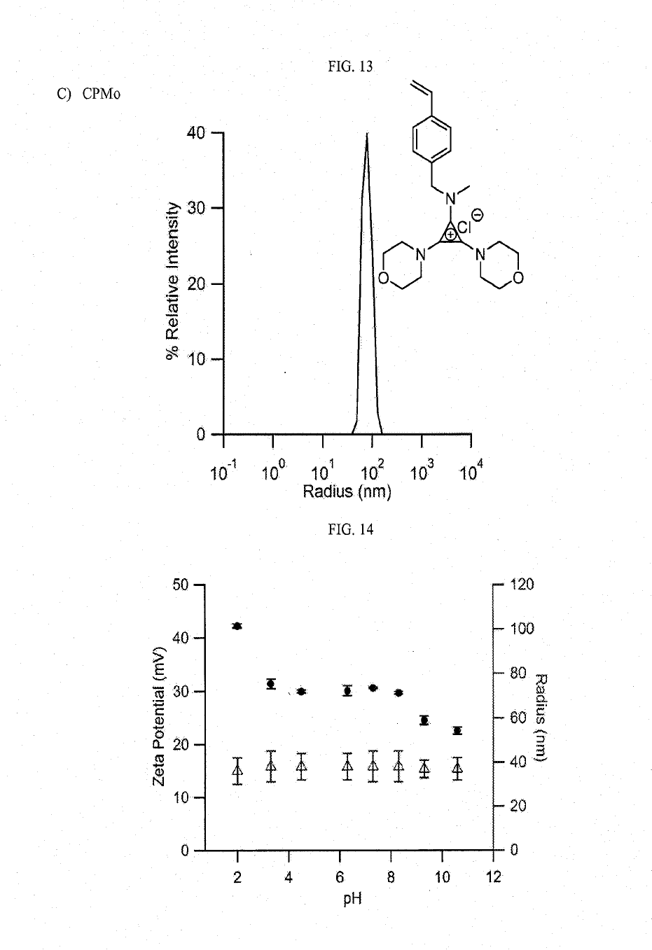

The disclosed method overcomes limitations of current non-viral nanoparticle-based technologies in several ways: (1) control over particle size and charge, with hydrated radii as small as about 10 nm to about 20 nm (up to greater than about 100 nm)^{1,2} and zeta potentials ranging from about +5 to about +50 mV′⁴ (2) a scalable, simple synthesis route to polymer/PNA complexes;⁵ (3) no harsh conditions required which can damage molecules;⁶ (4) avoids the use of emulsifiers or surfactants (5) advantage of modular functional groups to tune interactions with biomolecules to optimize binding and release equilibria;⁷ (6) avoids the use of amine-based cationic groups that modulate charge with about pH8 and are known to be cytotoxic.⁴ Examples of particles with modular functionality are shown in FIG. 13.

The ability to introduce a variety of R-groups which may vary solubility, steric and electrostatic interactions, and introduce potential functional handles demonstrates the modularity of the CPR monomers. The monomer structure, specifically the modular R-groups, correlate with particle diameter and surface charge. In addition, hydrophobic molecules may easily functionalize the interior of the particles with, for example, fluorescent dyes that are useful for imaging applications.

A distinguishing feature of these polymer-based systems for, preferably, gene therapy application is their synthetic versatility. The charge exuded by the polymers/nanoparticles can easily be tuned by adding a co-monomer at a certain ratio. Furthermore, the hydrophilicity can be altered in several ways, including through the incorporation of co-monomers or via the use of different R-substituents on the cyclopropenium monomers themselves. The molecular weight of the synthetic polymers can also be precisely dialed in, which affects their solution aggregation, and thus the final size of the resulting polyplexes with PNAs. The flexibility of these properties can enable the tuning of the transfection ability while maintaining low cytotoxicity. The incorporation of targeting moieties can be achieved by incorporating them into the monomer feed or through post-polymerization modification. Additionally, the particle synthesis provides the opportunity to tune the size of the nanostructures simply by changing the incorporation of the cyclopropenium monomer incorporation. Synthesized cationic particles are preferably in the size range of about 140 nm down to about 30 nm, which may be particularly useful for targeting different cell types or addressing various biological cargoes. Along these lines, synthesizing the particle in the presence of BCP stabilizers leads to particles displaying a more densely packed core surrounded by chains extending into solution forming a polymer corona. This corona might serve to protect, for example, a nucleic acid cargo from degradation or premature release in vivo.

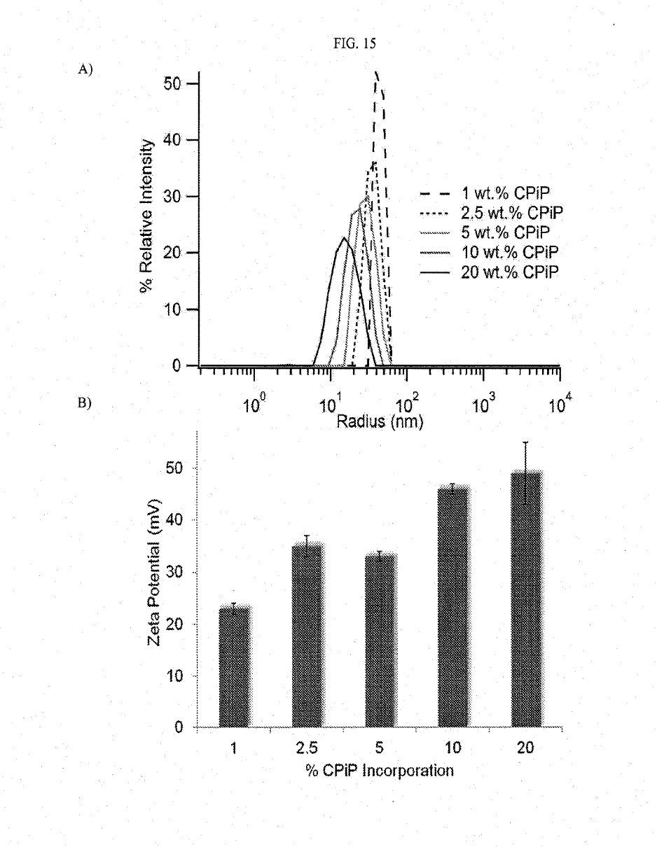

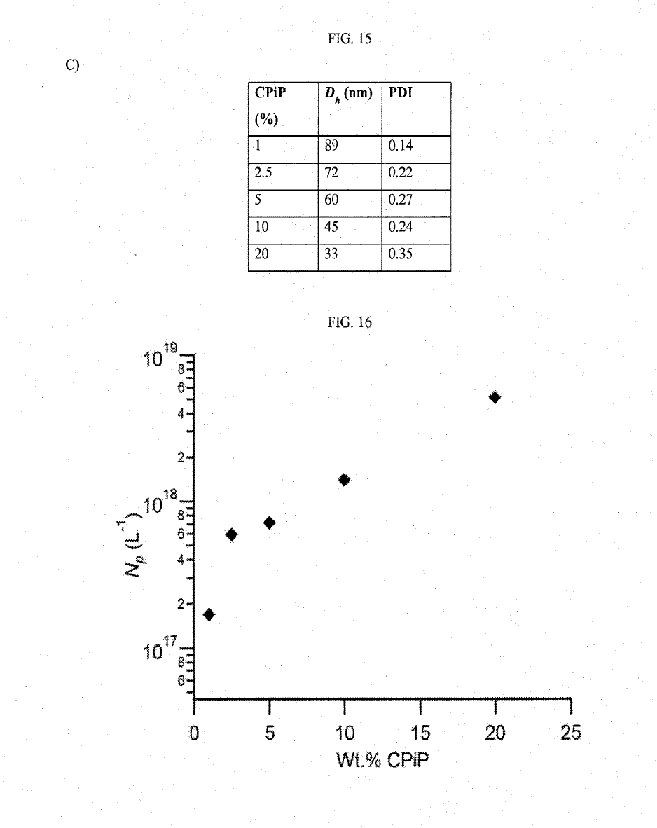

A preferred embodiment is directed to a process or method for synthesizing cationic nanoparticles containing cyclopropenium-based chemical moieties by combining a cyclopropenium-based chemical moiety, a co-monomer species, an initiator, and water to form a mixture; and heating the mixture. The cyclopropenium-based chemical moiety may be a CP-based monomer or block copolymer, which may be combined with a majority hydrophobic co-monomer species, preferably styrene, and solubilized with water. A water soluble radical initiator, such as for example, the water soluble, surface-active cationic azo initiator (V-50; 2,2′-azobis(2-methylpropionamidine) dihydrochloride) is added in an amount sufficient to initiate polymerization and heated to a temperature ranging from about 60° C. to about 80° C., preferably about 70° C. to form the inventive surface-charged CP-based nanoparticle. The initiator may be solubilized with the CP-based chemical moiety and co-monomer, or after solubilizing the CP-based chemical moiety and co-monomer. The weight ratio of the CP-based monomer (CPR) or block copolymer (BCP) to styrene (CPR or BCP:Styrene) determines the resulting nanoparticle size. (see, FIG. 15) By increasing the amount of CP-based monomer or block copolymer, the nanoparticle size gets smaller. The increase in monomer concentration leads to a faster polymerization rate and a smaller particle size. FIG. 16 shows that Np increases with incorporation, which is nearly exponential above 1%. The weight percent amount of CPR or BCP may be about 0.5% to about 20% relative to styrene in an amount of about 99.5% to about 80%. The combination of CPR or BCP equals about 10 wt % and water makes up the remaining approximately 90%, such that the CPR or BCP is sufficiently dilute.

Another embodiment is directed to the cationic cyclopropenium-based nanoparticles synthesized by the process or method of the invention described here. The preferred nanoparticles have a positively charged surface, where the functionalized interior is hydrophobic and exterior is hydrophilic. The nanoparticles are spherical or essentially spherical in shape, and have a tunable size preferably ranging from about 30 nm to about 100 nm. The nanoparticles in aqueous solution are not contaminated with any organic solvent. As described in the methods of synthesis, the nanoparticles are self-assembled into the particular geometry.

These surface-charged nanoparticles have a broad range of applications. As CP is a remarkably stable carbocation, the nanoparticles retain their charge over a wide pH range. The nanoparticle interior can be covalently functionalized with fluorescent dyes useful for biomedical applications, as well as the use of these nanoparticles as additives, gene-delivery vectors or carriers, and chromatographic separation, and the like. These applications resulting from the beneficial characteristics of the inventive nanoparticles also include but are not limited to drug delivery, ion-exchange resins, affinity chromatography (DNA, proteins), filtration/purification, immunoassays, enzyme stabilization, antimicrobial coatings/antibiofouling, organocatalyst supports, cosmetics, therapeutics, diagnostics, and the like. The versatility of CP-based monomers and BCPEs for the synthesis of surface-charged nanoparticles is particularly useful in that they may be functionalized with fluorescence. For example, a fluorescent tag may be covalently linked to the nanoparticle in the core of the particle versus outside or externally, thereby protecting the fluorescence.

Polymers offer a rich palate to be decorated with functional units in order to tune various properties, and to harness the collective interactions of the building blocks that can be exploited for technological advances. However, introducing functionality can alter the supramolecular interactions leading to unpredictable behavior. Non-conventional building blocks that are commonly overlooked in order to exploit organic materials in multiple applications ranging from energy storage/generation and biology. Materials based on cyclopropenium ions may be used as a means for transfection, such as for example, cancer cells not previously transfected including gastric carcinomas. It is contemplated that a label-free imaging technique may be used to track nanoparticles that only contain styrene and cyclopropenium ion monomers, without any other tags. The inventive nanoparticles have broad implications in biotechnology to study mechanisms of cell transfection and cell function using knock-down sequence strategies, among many other applications.

The compositions useful in the practice of the methods of the invention may be administered to a mammal by any means known in the art including, but not limited to oral or peritoneal routes, such as, for example, intravenous, intramuscular, intraperitoneal, subcutaneous, transdermal, airway (aerosol), rectal, vaginal and topical (including buccal and sublingual) administration.

### Administration Routes

For oral administration, the carriers or polymer/particle compositions, may generally be provided in the form of tablets or capsules, as a powder or granules, or as an aqueous solution or suspension. Oral tablets may include the inventive carrier or polymer/particle composition containing active ingredients mixed with pharmaceutically acceptable excipients, such as, for example, inert diluents, disintegrating agents, binding agents, lubricating agents, sweetening agents, flavoring agents, coloring agents, and preservatives. Suitable inert diluents include, but not limited to, sodium and calcium carbonate, sodium and calcium phosphate, and lactose, while corn starch and alginic acid are suitable disintegrating agents. Binding agents may include starch and gelatin, while a lubricating agent, if incorporated, will typically be magnesium stearate, stearic acid, or talc. If desired, the tablets may be coated with a material such as glyceryl monostearate or glyceryl distearate, to delay absorption in the gastrointestinal tract.

Capsules for oral use may include hard gelatin capsules in which the carrier and active ingredient composition is mixed with a solid diluent, and soft gelatin capsules where the composition is mixed with water or an oil such as peanut oil, liquid paraffin, or olive oil. For intramuscular, intraperitoneal, subcutaneous, and intravenous use, the compositions of the invention may typically be provided in sterile aqueous solutions or suspensions, buffered to an appropriate pH and isotonicity. Suitable aqueous vehicles include Ringer's solution and isotonic sodium chloride. The excipient may consist exclusively of an aqueous buffer (i.e., no auxiliary agents or encapsulating substances are present which might affect or mediate uptake of the polymer/particle complex containing the molecule of interest). Such substances include, for example, micellar structures, such as liposomes or capsids, as described below. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The inventive compositions comprising polycationic particles conjugated to molecules, such as biomolecules, may also be encapsulated formulations to protect the therapeutic (e.g. a dsRNA compound) against rapid elimination from the body, such as a controlled release formulation, including implants and microencapsulated delivery systems. Biocompatible, biodegradable polymers can be used, such as ethylene vinyl acetate, polyanhydrides, polyglycolic acid, collagen, polyorthoesters, and polylactic acid. Methods for preparation of such formulations will be apparent to those skilled in the art. The materials may also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT Publication No. WO 91/06309; and European Patent No. EP0043075.

The inventive compositions can also comprise a delivery vehicle, including liposomes, for administration to a subject, carriers and diluents and their salts, and/or can be present in pharmaceutically acceptable formulations. For example, methods for the delivery of nucleic acid molecules are described in Akhtar et al., Trends Cell Bio., 1992 May, 2(5): 139-144; Saghir Akhtar. Delivery Strategies For Antisense Oligonucleotide Therapeutics (CRC Press, Boca Raton, Fla., 1995); Maurer et al., Mol. Membr. Biol., 1999 January-March, 16(1): 129-140; Hofland, H. and Huang, eds. L., Oxender, D., & Post, L. (1999). Formulation Delivery of Nucleic Acids. In Handbook Experimental Pharmacology (Vol. 137, pp. 165-192). Berlin: Springer; and Lee et al., 2000, ACS Symp. Ser., 752, 184-192. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule.

The compositions can be administered to a mammalian by a variety of methods known to those of skill in the art, including, but not limited to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the therapeutic/vehicle combination is locally delivered by direct injection or by use of an infusion pump. Direct injection of the composition, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262.

Pharmaceutically acceptable formulations of the polymer/particle complexes may include salts of the compounds, e.g., acid addition salts, for example, salts of hydrochloric, hydrobromic, acetic acid, and benzene sulfonic acid. A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell. For example, pharmacological compositions injected into the blood stream should be soluble. Other factors are known in the art, and include considerations such as toxicity and forms that prevent the composition or formulation from exerting its effect.

Administration routes that lead to systemic absorption (i.e. systemic absorption or accumulation of drugs in the blood stream followed by distribution throughout the entire body), are desirable and include, without limitation: intravenous, subcutaneous, intraperitoneal, inhalation, oral, intrapulmonary and intramuscular. Each of these administration routes exposes the polymer/particle carrier to an accessible diseased cell, tissue, or tumor. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

A “pharmaceutically acceptable formulation” may be a composition or formulation, i.e., the polymer/particle complex comprising a desirable active ingredient, such as a therapeutic, nucleic acid, protein, or the like that allows for the effective distribution of the composition of the instant invention in the physical location most suitable for their desired activity.

Therapeutic compositions comprising surface-modified liposomes containing poly (ethylene glycol) lipids (PEG-modified, or long-circulating liposomes or stealth liposomes) may also be suitably employed in the methods of the invention. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

Therapeutic compositions may include a pharmaceutically effective amount of the desired compounds in a pharmaceutically acceptable carrier or diluent. Acceptable carriers or diluents for therapeutic use are well known in the pharmaceutical art, and are described, for example, in REMINGTON'S PHARMACEUTICAL SCIENCES, Mack Publishing Co. (A. R. Gennaro, Ed. 1985). For example, preservatives, stabilizers, dyes and flavoring agents can be provided. These include sodium benzoate, sorbic acid and esters of p-hydroxybenzoic acid. In addition, antioxidants and suspending agents can be used.

### Dosage or Effective Amounts

The quantity of an active agent for effective therapy will depend upon a variety of factors, including the type of disease, disorder, or condition, means of administration, physiological state of the patient, other mendicants administered, and other factors. Treatment dosages generally may be titrated to optimize safety and efficacy. Typically, relationships between dosage and its effect from in vitro studies initially will provide useful guidance on the proper doses for patient administration. Studies in animal models also generally may be used for guidance regarding effective dosages for treatment of the disease, disorder, or condition in accordance with the present invention. These considerations, as well as effective formulations and administration procedures are well known in the art and are described in standard textbooks, such as GOODMAN AND GILMAN'S: THE PHARMACOLOGICAL BASES OF THERAPEUTICS, 8th Ed., Gilman et al. Eds. Pergamon Press (1990) and REMINGTON'S PHARMACEUTICAL SCIENCES, 17th Ed., Mack Publishing Co., Easton, Pa. (1990), both of which are incorporated by reference herein in their entirety.

Typical therapeutic doses will be about 0.1 mg/kg of body weight to about 1.0 mg/kg of body weight of pure active ingredient. The does may be adjusted to attain, initially, a blood level of about 0.1 μM. A particular formulation of the invention may use a lyophilized form of an active ingredient, in accordance with well-known techniques. For instance, about 1 mg to about 100 mg of active ingredient may be lyophilized in individual vials, together with carrier and buffer compound, for instance, such mannitol and sodium phosphate. The active ingredient may be reconstituted in the vials with bacteriostatic water and then administered, as described elsewhere here. A pharmaceutically effective dose or amount is that dose required to prevent or inhibit, or modulate or increase depending on the particular disease, condition, or disorder, the symptoms or occurrence, treat by alleviating a symptom to some extent, preferably all of the symptoms of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between about 0.1 mg/kg body weight/day and about 100 mg/kg body weight/day of active ingredients is administered.

Dosage levels of the order of from about 0.1 mg to about 140 mg per kilogram of body weight per day may be useful in the treatment of conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

A therapeutic useful in the practice of the invention may comprise a single compound, or a combination of multiple compounds, whether in the same class of inhibitor (i.e. antibody inhibitor), or in different classes (i.e., antibody inhibitors and small-molecule inhibitors). Such combination of compounds may increase the overall therapeutic effect in inhibiting the progression of a fusion protein-expressing cancer. For example, the therapeutic composition may a small molecule inhibitor, or in combination with other inhibitors targeting a particular activity and/or other small molecule inhibitors. The therapeutic composition may also comprise one or more non-specific chemotherapeutic agent in addition to one or more targeted inhibitors. Such combinations have recently been shown to provide a synergistic tumor killing effect in many cancers. The effectiveness of such combinations in inhibiting a particular activity and tumor growth in vivo can be assessed.

### Articles of Manufacture

In another embodiment of the invention, an article of manufacture, orkit, containing materials useful for treating particular diseases and disorders associated with an active ingredient is provided. One embodiment relates to a kit that comprises a container comprising cationic nanoparticles comprising a cargo which may be an active ingredient, such as a nucleic acid, protein, peptide, enzyme, or compound, or a stereoisomer, tautomer, solvate, metabolite, or pharmaceutically acceptable salt or prodrug thereof. The kit may further comprise a label or package insert on or associated with the container. The package insert that is used refers to instructions customarily included in commercial packages of therapeutic products, that contain information about the indications, usage, dosage, administration, contraindications, and/or warnings concerning the use of such therapeutic products. Suitable containers include, for example, bottles, vials, syringes, blister pack, etc. The container may be formed from a variety of materials such as glass or plastic. The container may hold the cationic polymers and particles conjugated to or containing a specific cargo or active ingredient or a formulation which is effective for treating the condition that the active ingredient is known to prevent, inhibit, modulate, increase, and may have a sterile access port (for example, the container may be an intravenous solution bag or a vial having a stopper pierceable by a hypodermic injection needle). One exemplary active agent in the composition may be a nucleic acid, polynucleic acids, DNA, mRNA, siRNA, etc. The label or package insert indicates that the composition is used for treating the condition of choice, such as cancer. In addition, the label or package insert may indicate that the patient to be treated is one having a disorder such as a disease or condition that the active ingredient affects. In one embodiment, the label or package inserts indicates that the composition can be used to treat any particular disorder. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the composition and, if present, the second pharmaceutical formulation. For example, if the kit comprises a first composition comprising an active ingredient and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a composition of the inventive carrier with a first active ingredient contained within; and optionally (b) a second container with a second pharmaceutical formulation contained within, wherein the second pharmaceutical formulation comprises a second compound that preferably works advantageously together with the first pharmaceutical composition. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, syringes, and/or desired items that may assist with the use or administration of the pharmaceutical compositions.

In certain other embodiments where the kit comprises a cationic polymer/particle composition and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit is particularly advantageous when the separate components are preferably administered in different dosage forms (e.g., oral and parenteral), are administered at different dosage intervals, or when titration of the individual components of the combination is desired by the prescribing physician.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

Having now fully described the subject compositions and methods, it will be understood by those of ordinary skill in the art that the same can be performed within a wide and equivalent range of conditions, formulations and other parameters without affecting their scope or any embodiment thereof. All cited patents, patent applications, publications, and documents are fully incorporated by reference in their entirety.

### EXAMPLES

The following Examples of the invention are provided only to further illustrate the invention, and are not intended to limit its scope.

All materials were purchased from Aldrich and were used without further purification, except as noted below. Styrene and 1-pyrenemethyl methacrylate were passed over a column of neutral alumina to remove inhibitor prior to polymerization. Deuterated solvents for NMR were purchased from Cambridge Isotope Laboratories, Inc The CPR monomers and BCPEs were synthesized according to previously described procedures. (Jiang, Y.; Freyer, J. L; Cotanda, P.; Brucks, S. D.; Killops, K. L.; Bandar, J. S.; Torsitano, C.; Balsara, N. P.; Lambert, T. H.; Campos, L. M. Nat. Commun. 2015, DOI: 10.1038/ncomms6950)

### Example 1

### Emulsion Polymerization of Particles

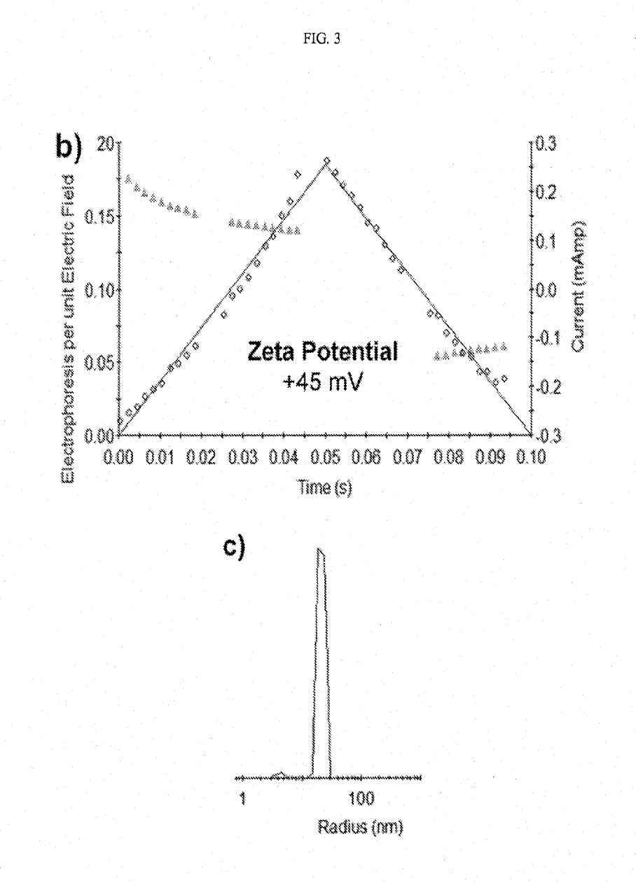

The emulsion polymerization of particles was executed as described here. The cyclopropenium monomer was weighed out into a vial. Styrene monomer, was added to the vial and the two monomers were mixed to dissolve the cyclopropenium monomer. In a separate vial, a water soluble azo initiator, 2,2′-azobis(2-methylpropionamidine) dihydrochloride, was dissolved in a small amount of water. Water was added to the vial containing monomers so that they constituted 10 wt. % of the total weight of the reaction. The initiator solution was added to the monomers, and the vial was vortexed to emulsify. The emulsion was added to a round bottom flask fitted with a condenser and stirbar, and the reaction mixture was stirred and sparged with inert gas for 10 minutes. The vessel was then sealed and heated at 70° C. for 6-18 hours. Particle size was determined by dynamic light scattering and scanning probe microscopy, scanning electron microscopy, or transmission electron microscopy. Electrophoretic potential measurements were conducted to determine the zeta potential of the particle solution (FIG. 3).

### Example 2

### DNA Complexation to the Particles

The particle solution was simply mixed with DNA at different ratios to match the approximate number of charged groups on the surface of the particle to the number of phosphate units in the DNA backbone. The extent of complexation was determined by gel electrophoresis and DLS/zeta potential measurements (FIG. 1B).

### Example 3

### Particle Synthesis with CPR Monomer

Particles were synthesized by following a general procedure that was scaled accordingly using 1-20 wt % monomer (relative to styrene), styrene, 2,2-azobis(2-methylpropionamidine) dihydrochloride (V-50), and water. The final solution was scaled to 10 g, with 10% monomer content. First, CPR monomer was dissolved in styrene, and initiator was dissolved separately in 1 mL of water. The remaining volume of water was added to the monomer solution, and the V-50 solution was finally added to the monomer suspension. The mixture was vortexed for 30 s. The solution was added to a two-neck flask fitted with a condenser and stir bar and was sparged with N for 10 min. The solution was stirred at 70° C. for 6-16 h.

### Example 4

### Particle Synthesis with BCPEs

Particles were synthesized by following a general procedure that was scaled accordingly using 5 wt % BCPE (relative to styrene), styrene, V-50, and water. The final solution was scaled to 10 g, with 10% monomer/BCPE content. First, BCPE was dissolved in styrene and initiator was dissolved separately in 1 mL of water. The remaining volume of water was added to the monomer solution, and the V-50 solution was finally added to the monomer suspension. The mixture was vortexed for 30 s. The solution was added to a two-neck flask fitted with a condenser and stir bar and was sparged with N for 10 min. The solution was stirred at 70° C. for 6-16 h.

### Example 5

### Particle Synthesis with Fluorescein O-Methacrylate

In a vial, CPiP (42 mg, 0.10 mmol) was dissolved in styrene (378 mg, 3.63 mmol). Fluorescein O-methacrylate (FMA) (20 mg, 0.05 mmol) was added to the vial and vortexed for 30 s. V-50 (11 mg, 0.04 mmol) was added with 3.5 mL of DI H₂O. The oil-in-water solution was vortexed for another 30 s. The solution was added to a two-neck flask fitted with a condenser and stir bar and was sparged with Ar for 10 min. The reaction was then heated at 70° C. for 24 h with stirring. The reaction mixture was cooled and dialyzed against methanol for 24 h to remove unreacted monomer.

### Example 6

### Particle Synthesis with 1-Pyrenemethyl Methacrylate

In a vial, CPiP (57 mg, 0.14 mmol) was dissolved in styrene (513 mg, 4.93 mmol). 1-Pyrenemethyl methacrylate (PMA) (10 mg, 0.033 mmol) of was added to the vial and vortexed for 30 s. V-50 (8 mg, 0.03 mmol) was added with 5 mL of DI H₂O. The oil-in-water solution was vortexed for another 30 s. The solution was added to a two-neck flask fitted with a condenser and stir bar and was sparged with Ar for 10 min. The reaction was then heated at 70° C. for 24 h with stirring. The reaction mixture was cooled and dialyzed against methanol for 24 h to remove unreacted monomer.

### Example 7

### Congo Red Dye Adsorption by CPR Monomer Particles and BCPE Particles

Congo Red dye was dissolved in deionized water at 50, 40, 30, 20, and 10 mg L⁻¹ concentrations to establish a calibration curve (λₘₐₓ 498 nm). Congo Red (5 mL of 50 mg L⁻¹) was incubated with 5 mL of 50 mg L⁻¹ of either 5CPiP or 5iPBCP particles. These solutions were vortexed for 10 min and centrifuged (3750 rpm, 15 min), and the absorbance of the supernatant was measured spectrophotometrically. Dye concentrations were calculated from the calibration curve.

### Example 8

### Congo Red Dye Adsorption by CPR Monomer Particles and BCPE Particles

¹H NMR spectra were recorded in CDCl₃ on a Bruker 300 MHz spectrometer. Chemical shifts are given in ppm relative to the signal from residual nondeuterated solvent.

Particle size, polydispersity, and electrophoretic mobility were measured using a Möbiuζ dynamic light scattering instrument and analyzed using Dynamics software from Wyatt Technology (Santa Barbara, Calif.). Particle size and polydispersity were calculated via the regularization fit of the correlation function of the quasi-elastic light scattering (QELS) data. Each measurement contained 10 acquisitions, and the reported radii or diameters are the average of three measurements. The zeta potential was calculated according to the Smoluchowski approximation, and reported values are the averaged result of five acquisitions from each of the 31 detectors in the massively parallel phase amplitude light scattering (MP-PALS) detector array. Measurements were run in Milli-Q water at neutral pH unless otherwise noted. Samples were passed through a 1.6 μm glass filter (Whatman) prior to measurement to remove only large aggregates and dust.

Scanning electron microscopy (SEM) was performed on a JEOL7001FLV at 3.0-10.0 keV. Particles were deposited on a silicon wafer from solution and imaged without sputtered metal coating. Particle sizes measured by SEM were determined using Image-J software by manually counting at least 50 particles. Centrifugation was performed on an Allegra 6R Centrifuge. UV-vis measurements were performed on a Shimadzu UV-1800 or a Jasco V-650 spectrophotometer, and fluorescence measurements were made on a Jasco FP-8300 (Easton, Md.). The samples were measured in optical grade methanol.

### Example 9

### Cationic Latex Particles Prepared with Different CP Monomers

Stable and polymerizable building blocks based on the aromatic cyclopropenium ion, where the formal charge is on carbon but extensively delocalized (30 2015 article) were developed. The use of isopropyl-, cyclohexyl-, and morpholine-functional CP monomers (CPiP, CPCy, and CPMo, respectively) to relate molecular structure to nanoparticle size and stability when they are copolymerized with styrene. Particles were prepared by standard emulsion polymerization with V-50 as the water-soluble radical initiator, simply mixing styrene with either a CPR monomer or BCPE, without additional surfactant. TABLE 1 summarizes the reaction conditions and properties of the synthesized cationic latex particles prepared with different CP monomers.

TABLE 1

| latex | wt % CP^{a} | Dₕ^{b} (nm) | PD^{b} | ζ-potential (mV) | Dₙ^{c} (nm) |
|---|---|---|---|---|---|
| styrene | 0 | 337 | 0.44 | 16 + 6 | 214 + 82 |
| 1CPiP | 1 | 89 | 0.13 | 23 + 1 | 76 ± 7 |
| 2.5CPiP | 2.5 | 70 | 0.16 | 35 ± 2 | 58 ± 5 |
| 5CPiP | 5 | 60 | 0.18 | 37 ± 1 | 39 ± 6 |
| 10CPiP | 10 | 48 | 0.22 | 46 + 1 | 36 ± 3 |
| 20CPiP | 20 | 34 | 0.28 | 54 ± 2 | 33 ± 4 |
| 5CPCy | 5 | 61 | 0.25 | 44 ± 1 | 53 ± 4 |
| 5CPMo | 5 | 100 | 0.21 | 48 + 1 | 85 ± 7 |

^{a}Determined from the monomer feed, relative to styrene.
^{b}Hydrodynamic diameter and polydispersity (PD) determined by DLS.
^{c}Particle diameter determined by SEM.

### Example 10

### Controlling Particle Size by Adjusting Amount of CP Monomers

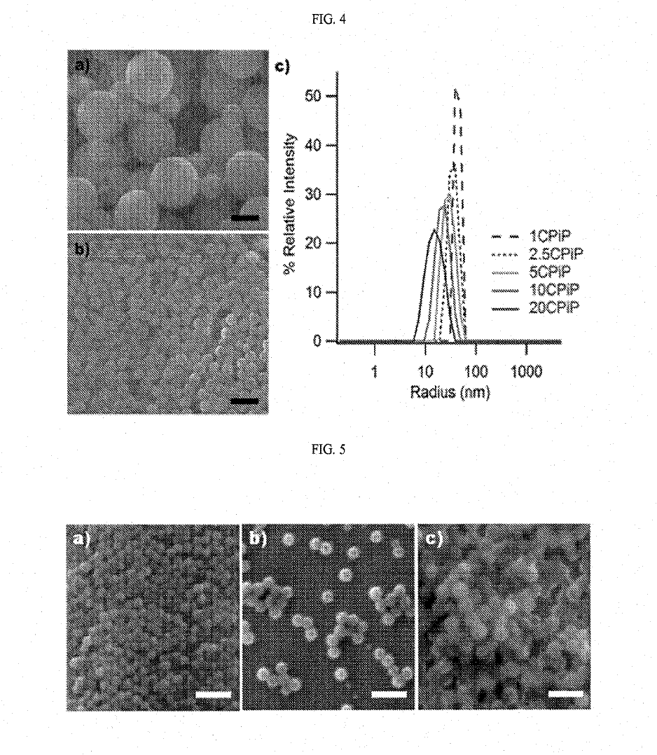

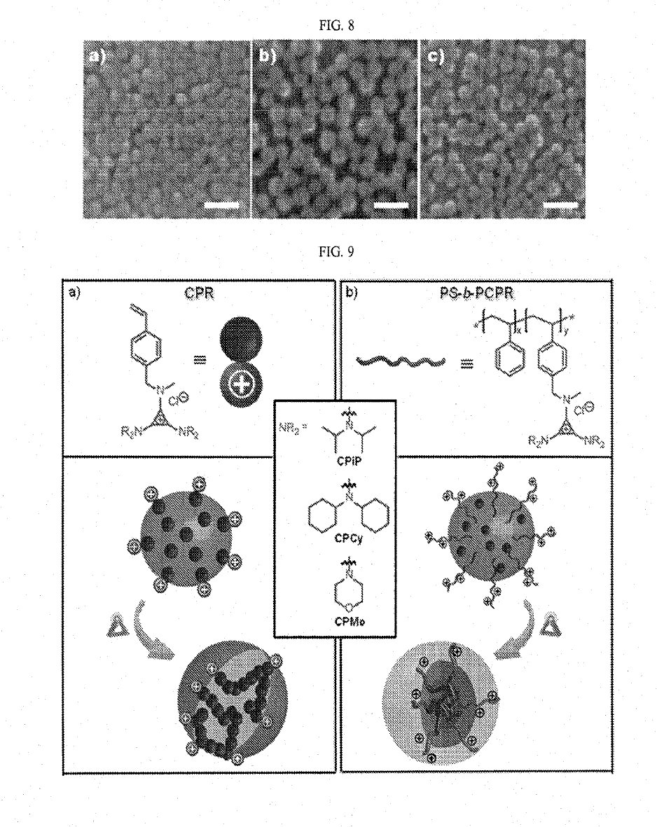

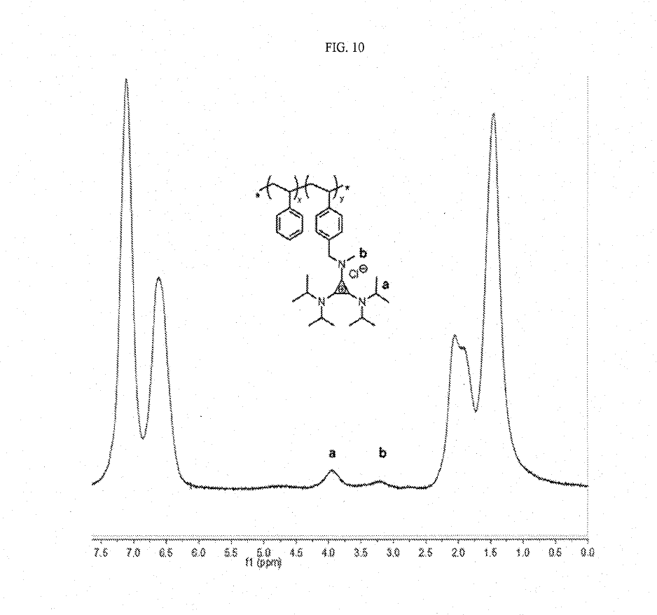

CPiP was incorporated into the monomer feed in an amount from about 1 weight percent (wt %) to about 20 wt %. At only 1 wt % CPiP (denoted 1CPiP) relative to styrene, the particle size and polydispersity were dramatically reduced, as compared with particles synthesized in the absence of CP (FIG. 4A,B). Incorporation of 1 wt % CPiP over the styrene-only particles resulted in an increase in ζ-potential. Without the CP comonomer, the charges conferred by the V-50 initiator stabilized the PS latex leading to large particles with a bimodal size distribution and a lower ζ-potential (FIG. 4A). Increasing feed of CPiP was found to affect particle size and polydispersity as determined by DLS (FIG. 4C). Particle size decreased as CP incorporation increased, demonstrating that the CPR functional monomer enabled the formation of particles as small as 34 nm in diameter in the case of 20CPiP. At loadings higher than 20 wt %, the styrene-CP copolymers became more water-soluble and no longer formed stable latex particles. The ζ-potential increased with higher CP loading, which corresponds to greater coverage of the particle surface with a cationic charge. Thus, even the smallest nanoparticles are electrostatically stabilized and bear significant charge: ca. 50 mV. Full conversion of CPiP was monitored by NMR, where the protons from unreacted monomer were absent from the region between c. 5.8 ppm and 5.3 ppm, indicating that they were fully copolymerized with styrene (FIG. 9). Between 5.0 ppm and 3.0 ppm of the ¹H NMR spectrum, broad peaks corresponding to distinct protons from copolymerized CPiP were visible. The nanoparticle size and surface charges may be precisely modified by viable and scalable synthetic strategies with these monomers.

### Example 11

### Role of CPR Moieties in the Formation of Nanoparticles

Monomers comprising cyclohexyl and morpholine functional groups, loaded at a constant weight percent, were also investigated as emulsion stabilizers. In the case of CPCy, the particles synthesized with 5 wt % comonomer displayed a size (60 nm, TABLE 1) similar to that of 5CPiP. However, for CPMo, the most hydrophilic monomer, particles synthesized with 5 wt % feed had slightly larger hydrated diameters of 100 nm. The increase in diameter is attributed to the greater hydrophilicity of CPMo, which lead to a small amount of monomer to be partitioned into the aqueous phase, yielding larger oil-in-water droplets prior to polymerization. It is particularly noteworthy that all three of the CPR monomers yielded nanoparticles with a monomodal size distribution (TABLE 1 and FIG. 5).

### Example 12

### Stability of Cationic Charge on CP Particles

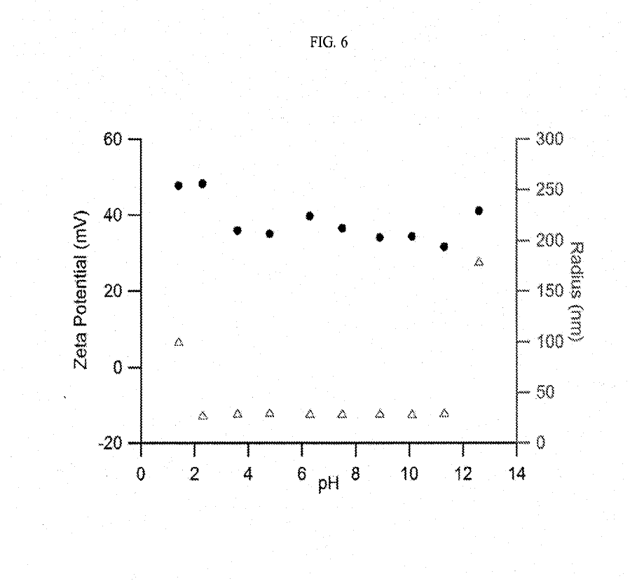

For those applications that require a persistent, stable cationic charge, the cationic moiety must remain positively charged over a wide pH range and that the particles resist flocculation. (Wang, Y.-J.; Qiao, J.; Baker, R.; Zhang, J. Chem. Soc. Rev. 2013, 42, 5768; Zeng, Z.; Patel, J.; Lee, S.-H; McCallum, M.; Tyagi, A.; Yan, M.; Shea, K. J. J. Am. Chem. Soc. 2012, 134, 2681). Cyclopropenium moieties have been shown to be stable. To examine the stability of cationic charge on the CP particles, ζ-potential and particle size were measured as a function of pH for 5CPiP (FIG. 6). The ζ-potential of the particles remained above 30 mV for the entire pH range from pH 1.4 to 12.6. This electrostatic stability is in stark contrast to typical amine-based cationic systems, which are neutralized above the pKₐ of the amine. (Voorn, D.-J.; Ming, W.; van Herk, A. M. Macromolecules 2005, 38, 3653). Furthermore, the CP particles remained stable over most of the pH range, as indicated by the constant hydrodynamic radius—indicative of no aggregation or degradation. However, at the extreme values of the pH range (12.6 and 1.4), the particles were destabilized and aggregates were observed by DLS. These broadly pH-stable cationically-charged nanoparticles were found to be stable at room temperature and 1 atmospheric pressure for at least one month.

### Example 13

### Loading the Interiors of Particles with Fluorescent Molecules for Diagnostic Imaging

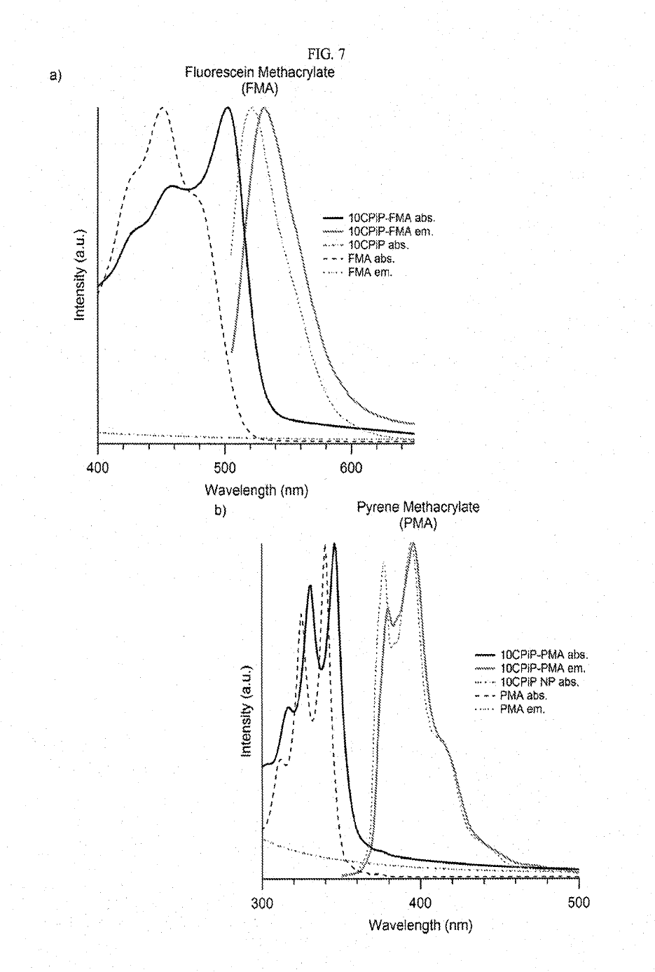

Emulsion polymerization allows loading the interior of particles with, for example, either a therapeutic agent to facilitate delivery of a drug payload (Elsabahy, M.; Shrestha, R.; Clark, C.; Taylor, S.; Leonard, J.; Wooley, K. L. Nano Lett. 2013, 13, 2172) or fluorescent molecules for diagnostic imaging. (Akbulut, M.; Ginart, P.; Gindy, M. E.; Theriault, C.; Chin, K. H.; Soboyejo, W.; Prud'homme, R. K. Adv. Funct. Mater. 2009, 19, 718). Hydrophobic fluorescent molecules were readily incorporated into the CP nanoparticle interior. To minimize leaching of the dye resulting from weak hydrophobic interactions (Yin Win, K.; Feng, S.-S. Biomaterials 2005, 26, 2713), the fluorescent monomers were covalently linked into the particle framework by using a polymerizable dye. Commercially available fluorescein O-methacrylate (FMA) and 1-pyrenemethyl methacrylate (PMA) were chosen for their distinct excitation and emission spectra. Each of the fluorophore monomers was included in the emulsion polymerization mixture at low molar equivalents (1 mol % FMA, 0.6 mol % PMA) along with CPiP (at 10 wt %) to ensure that the particle diameters remained below 50 nm. This is a critical size regime for biomedical applications. The particles were dialyzed against methanol after polymerization to remove any unreacted monomer. The emission and absorption spectra for each of the fluorescently labeled particles can be seen in FIG. 7. For particles without dye, no absorption above 300 nm was observed (FIG. 7), as opposed to the purified dye-containing particles with λₘₐₓ at 346 nm and 502 nm for PMA and FMA, respectively. Distinct bathochromic shifts in the absorption and emission spectra for both FMA and PMA particles, relative to the free monomer, were observed, indicating aggregation of the dyes within the particle interior. (Sauer, M.; Hofkens, J.; Enderlein, J. Handbook of Fluorescence Spectroscopy and Imaging: From Single Molecules to Ensembles; Wiley: Weinheim, Germany, 2011.) The fluorescent particles maintained their small diameter and highly positive ζ-potentials—45 nm and 48 mV for FMA and 38 nm and 49 mV for PMA—demonstrating their tolerance to functionalization. The ability to covalently link fluorescent molecules within the interior of the particles may be useful for in vitro imaging and diagnostics and can be generalized to other hydrophobic molecules of interest. (Peng, H.-S.; Chiu, D. Chem. Soc. Rev. 2015, in press)

### Example 14

### Cationic Latex Particles Prepared with Different CP BCPEs

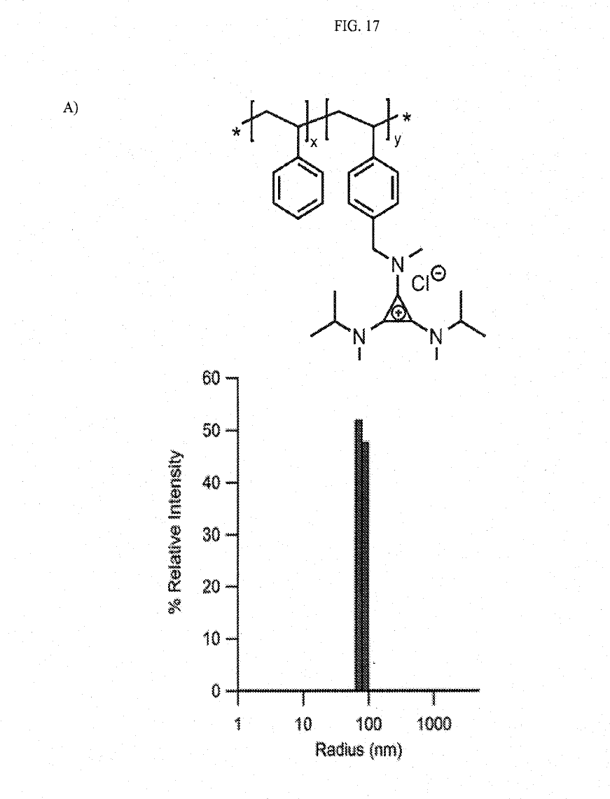

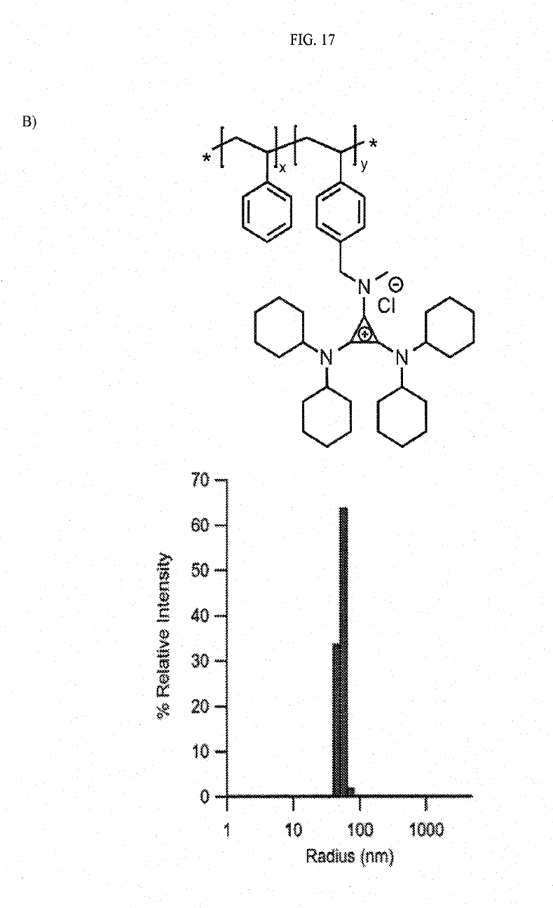

In addition to the CPR monomers, amphiphilic CP BCPEs were used as electrosteric particle stabilizers in the surfactant-free emulsion polymerization of styrene and were directly compared to their monomer analogues. Although the BCPEs were not covalently bound to the particles, the diffusion coefficients for BCP stabilizers were orders of magnitude lower than small molecule surfactants due to the hydrophobic block of the BCP being embedded in the particle interior (Riess, G.; Labbe, C. Macromol. Rapid Commun. 2004, 25, 401). Particles with distinct functional corona were synthesized with polystyreneb-poly(cyclopropenium) [PS-b-PCPR] BCPEs containing cyclohexyl-, isopropyl-, and morpholine-functional CP (CyBCP, iPBCP, and MoBCP, respectively). (see, FIG. 17) Each of the BCPEs was synthesized via reversible addition—fragmentation chain transfer (RAFT) polymerization to contain 30 mol % CP relative to PS (TABLE 2) (Jiang, Y.; Freyer, J. L; Cotanda, P.; Brucks, S. D.; Killops, K. L.; Bandar, J. S.; Torsitano, C.; Balsara, N. P.; Lambert, T. H.; Campos, L. M. Nat. Commun. 2015, DOI: 10.1038/ncomms6950). The BCPEs were not directly soluble in water, so they were dissolved in the styrene monomer before the addition of water to create the emulsion. The amphiphilic nature of this BCPE anchors PS block to the particle core (Burguière, C.; Pascual, S.; Bui, C.; Vairon, J.-P.; Charleux, B.; Davis, K. A.; Matyjaszewski, K.; Bétremieux, I. Macromolecules 2001, 34, 4439) with the CP block extending into solution to stabilize the particle (FIG. 9B).

The BCPEs were added to the emulsion at 5 wt % relative to styrene, which translates to less than 1 mol % of CP. Even at this low incorporation of the BCPE, stable particles were formed. The particles were characterized by DLS, electrophoretic mobility, and SEM (FIG. 8). Particles formed from 5CyBCP had the largest diameter at 101 nm and highest ζ-potential at 47 mV of the BCPEs tested. All three BCPs yielded particles with similar sizes and ζ-potentials, with a relatively narrow size distribution. The dramatic difference in particle diameter from DLS and SEM measurements suggests that these nanoparticles possess a polymer corona extending from the particle surface. (Riess, G.; Labbe, C. Macromol. Rapid Commun. 2004, 25, 401). Nanoparticles derived from BCPEs were found to be structurally distinct from monomer-derived particles, with the latter having charge localized at the surface, as opposed to the cationic blocks that extend into solution (FIG. 9). The diffuse corona, akin to polymer micelles (Navarro, G.; Pan, J.; Torchilin, V. P. Mol. Pharmaceutics 2015, 12, 301), could be beneficial for anchoring molecules such as enzymes (Cao, L. Curr. Opin. Chem. Biol. 2005, 9, 217) and RNA (Forbes, D. C.; Peppas, N. A. ACS Nano 2014, 8, 2908) to the particle surface or for enhancing cellular uptakein vitro. (Zhao, F.; Zhao, Y.; Liu, Y.; Chang, X.; Chen, C.; Zhao, Y. Small 2011, 7, 1322). Additionally, the particles formed through stabilization with CP BCPEs were determined to be larger than those formed by CP monomers. This can be understood from the relative number of BCPE molecules present, as there are fewer to stabilize particles. Conversely, a larger number of CP monomer molecules can achieve better surface coverage, which more effectively stabilizes the droplet/particle interface, leading to smaller particles overall. The number of and size of nanoparticles formed may be adjusted.

TABLE 2

| | MW^{a} | | | | | ζ- | |
|---|---|---|---|---|---|---|---|
| | (kg/ | mol % | | Dₕ^{b} | | potential | Dₙ^{c} |
| CPBCP | mol) | CP^{a} | latex | (nm) | PD^{b} | (mV) | (nm) |
| iPBCP | 30 | 30 | 5iPBCP | 94 | 0.15 | 39 ± 2 | 72 ± 8 |
| CyBCP | 50 | 30 | 5CyBCP | 101 | 0.09 | 47 ± 1 | 89 ± 8 |
| MoBCP | 45 | 30 | 5MoBCP | 94 | 0.17 | 41 ± 2 | 76 ± 9 |

^{a}Determined by ¹H NMR.
^{b}Hydrodynamic diameter and polydispersity (PD) determined by DLS.
^{c}Particle diameter determined by SEM

### Example 15

### Comparison of Binding Efficiency of Monomer- and BCPE-Based Particles

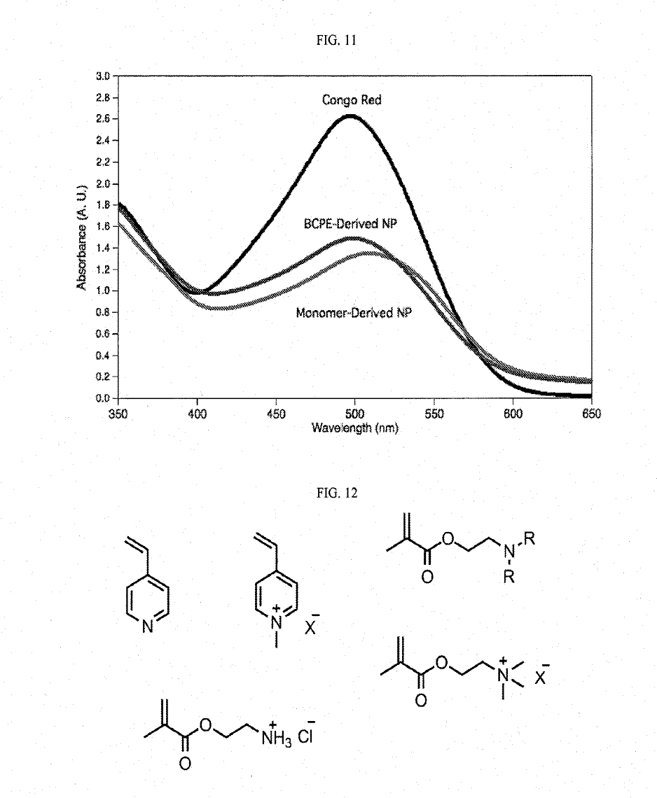

The binding efficiencies of the CP monomer- and BCPE-based nanoparticles were compared by incubating them with anionic dye, Congo Red. Without optimization, both 5CPiP and 5iPBCP exhibited greater than 40% binding efficiency, with the monomer-derived particles slightly more effective (FIG. 11). While these dye removal properties were not as potent as previous nanoparticle reports (Das, S. K.; Khan, M. M. R.; Parandhaman, T.; Laffir, F.; Guha, A. K.; Sekaran, G.; Mandal, A. B. Nanoscale 2013, 5, 5549; Burakowska, E.; Quinn, J. R.; Zimmerman, S. C.; Haag, R. J. Am. Chem. Soc. 2009, 131, 10574), this system enables a comparison of nanoparticle formulations with respect to interfacial molecular interactions. Access to a variety of particle sizes and properties based on the nature of the CP-stabilizer further reinforces the modularity of this system.

The content of all patents, patent applications, published articles, abstracts, books, reference manuals, and abstracts, as cited here are incorporated by reference in their entireties to more fully describe the state of the art to which the disclosure pertains.

All of the features disclosed in this specification and the appendix may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variations that fall within the scope of the appended claims.

### REFERENCES

(1) Beringer, M.; Scaria, P.; Woodle, M., Engineered Tunable Nanoparticles for Delivery of Therapeutics, Diagnostics, and Experimental Compounds and Related Compositions for Therapeutic Use. WO 2009108822A1, 2009.
(2) Muller, A.; Freitag, R.; Synatscke, C.; Majewski, A.; Jerome, V.; Schalon, A., Non-viral transfection systems. W02012156058A1, 2012.
(3) Vogel, J. M.; Boschetti, E., Compositions and Methods for Gene Therapy. W00172280A2 2001.
(4) Gupta, K.; Kumar, P.; Swami, A.; Pathak, A., Novel Crosslinked PEI Nanoparticle-based Transfection Agents for Delivery of Biomolecules. US 20100029750A1, 2010.
(5) Hubbell, J.; O'Neil, C.; Reddy, S.; Swartz, M.; Velluto, D.; Vlies, A.; Simeoni, E., Nanoparticles for Immunotherapy. U520100055189, 2010.
(6) Vanderhoff, J.; El-Aasser, M.; Ugelstad, J., Polymer Emulsification Process. U.S. Pat. No. 4,177,177, 1979.
(7) Hedrick, J.; Fukushima, K.; Yang, Y., Biodegradable Polymers, Complexes Thereof for Gene Therapeutics and Drug Delivery, and Methods Related Thereto. US20110151566A1, 2011.
(8) Prakash, S.; Malhotra, M., Non-viral Nanoparticle-Based Delivery System. U520130337067A1, 2013.
(9) Wang, L.-F.; Sung, K.-H.; Lo, Y.-L., Polysaccharide-grafted Polyethyleneimine as a Gene Carrier. US 20130030119A1, 2013.
(10) Seymour, L.; Ulbrich, K., Modification of Nucleic Acid Vectors with Polymers Comprising Charged Quaternary Amino Groups. WO 2010067041A1, 2010.
(11) Wendorff, J.; Aigner, A.; Dersch, R.; Nobel, S.; Rudisile, M., Non-viral Transfection Agent. WO 2010086406 A1, 2010.
(12) Yoon, H.; Dell, E. J.; Freyer, J. L.; Campos, L.; Jang, W.-D. Polymer 2014, 55, 453.
(13) Elsabahy, M.; Wooley, K. Chemical Society Reviews 2012, 41, 2545.
(14) Guo, X.; Huang, L. Accounts of Chemical Research 2012, 45, 971.
(15) Chu, Y.; Masoud, M.; Gulilat, G., Transfection reagents. U.S. Pat. No. 7,166,745 B1, 2007.
(16) Wullner, U.; Neef, I.; Tur, M.; Barth, S. Recent Patents on Anti-Cancer Drug Discovery 2009, 4, 1.
(17) Mathiowitz, E.; Jong, Y.; Boekelheide, K., Polymeric Gene Delivery System. WO 9524929A2, 1995.
(18) Chu, C.-c.; Mutschler-Chu, M.; Song, H.; Liu, B.; Gomurashvili, Z., Biodegradable Cationic Polymer Gene Transfer Compositions and Methods of Use. WO 2009015143A1, 2009.
(19) Campos, L. M.; Lambert, T. H.; Bandar, J., Cyclopropenium Polymers and Methods for Making the Same. WO 2014022365 A1, 2014.
(20) Lambert, T. H.; Campos, L. M.; Bandar, J., Cyclopropenium Polymers and Methods for Making the Same. WO 2014022365 A1, 2014.

1. A process for synthesizing cationic nanoparticles containing cyclopropenium-based chemical moieties comprising:
combining a cyclopropenium-based chemical moiety, a co-monomer species, an initiator, and water to form a mixture; and
heating the mixture.

2. The process of claim 1, wherein the synthesis occurs by oil-in-water emulsion polymerization.

3. The process of claim 1, wherein the synthesis occurs by water-in-oil emulsion polymerization.

4. The process of claim 1, wherein the synthesis occurs by a seeded emulsion polymerization.

5. The process of claim 1, wherein the cyclopropenium is contained in linear polymers that are used to stabilize the emulsion and incorporated into the particles.

6. The process of claim 1, wherein the cyclopropenium is contained in a branched or dendritic polymeric architecture that is used to stabilize the emulsion and incorporated into the particles.

7. The process of claim 1, wherein the cyclopropenium-based moiety is a cyclopropenium-based monomer or a cyclopropenium-based block copolymer.

8. The process of claim 7, wherein the co-monomer species is selected from the group consisting of styrenic, acrylic, and methacrylic.

9. The process of claim 7, wherein the mixture further comprises a multivalent crosslinking component.

10. The process of claim 1, wherein the initiator is selected from the group consisting of a thermal initiator, a photoinitiator, and a redox initiator.

11. The process of claim 10, wherein the thermal initiator is an azo (AIBN, V-50) or peroxide (K2S208).

12. The process of claim 10, wherein the photoinitiator is 2,2-Dimethoxy-2-phenylacetophenone (DMPA) or benzophenone.

13-24. (canceled)

25. A method for synthesizing cationic polymers or block copolymers (BCPs) containing cyclopropenium moieties.

26. The process of claim 25, wherein the cyclopropenium polymer is linear, block, random, alternating, or branched.

27. The process of claim 25, wherein the cyclopropenium monomer is copolymerized with styrenic, acrylic, methacrylic, anhydride, or other monomer groups.

28. The process of claim 25, wherein the polymer has other polymers grafted.

29-33. (canceled)

34. The process of claim 1, wherein the synthesis occurs by surfactant-free emulsion polymerization.

35. A cationic nanoparticle synthesized by the process of claim 1.

36. A method of using the cationic nanoparticle of claim 35 in an application selected from the group consisting of: biomedical, diagnostic, gene-delivery, drug delivery, chromatographic separation or isolation, ion-exchange or affinity chromatography, filtration, purification, immunoassays, enzyme stabilization, antimicrobial coatings, antibiofouling, organocatalyst supports, cosmetics, therapeutics, and the like.
